(19) United States
(12) Patent Application Publication
(10) Pub. No.: US 2011/0287061 A1
(43) Pub. Date: Nov. 24, 2011

(54) Dietary composition and method for promoting healthy hair growth and melanogenesis

(76) Inventor: Cathy Beggan, Sparta, NJ (US)

(21) Appl. No.: 12/800,684

(22) Filed: May 20, 2010

(51) Int. Cl. A61K 9/00; A61P 17/14; A61K 38/44

(52) U.S. Cl. 424/400; 424/94.4

(57) ABSTRACT

A dietary composition for promoting melanogenesis in hair of a user comprising approximately 5,000 IU per day of catalase, a synergistic core component, 5-α-reductase inhibitors, an antioxidant component, and approximately 200 mg per day of L-tyrosine. Preferably, the synergistic core component comprises copper, vitamin B6, biotin, para-aminobenzoic acid, folic acid, zinc, and pantothenic acid. Further, 5-α-reductase inhibitors preferably comprise Saw palmetto, phytosterols, and nettle root extract. The antioxidant component preferably comprises horsetail, Fo-Ti, chlorophyll, and barley grass.

### FIELD OF INVENTION

The present invention relates generally to a dietary composition and method of combating graying of mammalian hair. In particular, the dietary composition according to the present invention comprises a combination of catalase, a synergistic core component, 5α-reductase inhibitors, an antioxidant component, and L-tyrosine for retarding, preventing, and/or reversing the graying of hair of a user.

### BACKGROUND, OF THE INVENTION

Hair is a keratin-containing filament that grows from the dermis of the skin. Found exclusively in mammals, hair often refers to two distinct structures: (1) the hair follicle; and (2) the shaft. The hair follicle is an organ located in the dermis of the skin. The main function of the hair follicle is to maintain stem cells to produce hair shaft. On the other hand, the hair shaft is always external to the skin. Roughly three components constitute the hair shaft. The first component, the cuticle, is the outermost structure comprising flat cells arranged in a scale-like orientation. The cuticle covers the second component, the cortex, which contains keratin bundles. The third component, the medulla, is located near or at the center of the hair shaft.

The hair is known to serve various purposes. From a practical point of view, the hair functions to protect the skin from external stimuli such as foreign objects and UV rays. Additionally, the hair serves as a sensory means due to nerve endings that are attached to each hair follicle. Furthermore, the hair has an ornamental function especially in humans. Hair on the head, due to its prominence, has great social significance as a sign of youth, vitality, and sexuality; People having voluminous and lustrous hair on their head are often regarded as young, attractive, and healthy. With age, hair tends to lose its volume, sheen and color, resulting in gray hair having thin and brittle appearance. This transformation is often regarded as a sign of old age, ill health, and bodily decline. As today's society creates significant pressure on individuals to stay young and healthy, many people go to great lengths to combat gray hair.

Unfortunately, there are currently very limited options available. As the graying of hair is generally considered to be irreversible, most individuals with gray/white hair can either choose to do nothing or mask their gray hair with coloring and/or bleaching. However, applying coloring and/or bleaching has its disadvantages.

One such disadvantage is the nuisance of regular touch up of colored/bleached hair. This is necessary in order to avoid undesirable gray roots. Furthermore, coloring/bleaching could be messy and its outcomes could be at times unpredictable, especially when it is self-applied. Additionally, coloring/bleaching could pose health risks to users. For example, irritating chemicals used in coloring/bleaching compositions cause allergic reactions and eye irritation in some cases. Moreover, certain ingredients in coloring/bleaching compositions may penetrate human and animal skin and cause health risks. Such ingredients include, but are not limited to 4-methoxy-m-phenylenediamine, 4-chloro-m-phenylenediamine, 2,4-toluenediamine, 2-nitro-p-phenylenediamine, and 4-amino-2-nitrophenol. These ingredients have been reported to cause cancer in at least one animal species in lifetime feeding studies. (U.S. Food and Drug Administration, Cosmetics: Hair Dye Products, (Nov. 1, 1997)). Many researchers have attempted to determine whether regular application of hair color/bleach causes cancer or other health problems. However, these findings remain inconsistent, and thus, potential health risks posed by coloring/bleaching are unknown. Thus, there is a need to provide a safer and more convenient alternative to coloring/bleaching.

One alternative is an orally administered dietary supplement to retard, prevent, and/or reverse graying of hair. For example, Nelson (U.S. Pat. No. 6,149,933) has proposed a dietary supplement that is useful for this purpose. Nelson's supplement comprises various ingredients that are generally known to be beneficial to hair including copper, vitamin B6, para-aminobenzoic acid (PABA), and pantothenic acid or salts thereof. However, these ingredients may not achieve their effects on gray hair to their fullest potential, as the ingredients are unaccompanied by suitable agents to amplify the beneficial effects on hair.

The key to developing a more potent composition lies in the mechanism of graying of hair. Graying of hair can be attributed to a gradual decline in melanogenesis, the process of pigment production. Although the exact mechanism of action is not known, hydrogen peroxide (H₂O₂) has been suggested to be the key element in the impairment of melanogenesis. H₂O₂ is a byproduct of oxygen metabolism and a major contributor to oxidative damage due to its strong oxidizing capacity. Under normal aerobic conditions, approximately 4% of the oxygen metabolized by mitochondria is converted to harmful superoxide ion, which can subsequently be converted to H₂O₂ and other highly reactive species. In moderate amounts, H₂O₂ plays an important role in biological functions, e.g., in immune system response. (Gregory E. Conner, et al., Lactoperoxidase and Hydrogen Peroxide Metabolism in the Airway, American Journal of Respiratory and Critical Care medicine, 166; S57-S61 (2002)). (Balázs Rada, et al., Oxidative Innate Immune Defenses by Nox/Duox Family NADPH Oxidases, Contributions to Microbiology, 15; 164-187 (2008)). However, when excess H₂O₂ accumulates, cells and their components incur oxidative damage. Specifically to graying of hair, excess H₂O₂ oxidatively damages tyrosinase, the key enzyme of melanogenesis. The damage could irreversibly deactivate the function of tyrosinase. Furthermore, accumulation of H₂O₂ ultimately leads to apoptosis (“cell death”) of hair follicle melanocytes, cells that perform melanogenesis. (J. M. Wood, et al., Senile Hair Graying: H₂O₂-mediated Oxidative Stress Affects Human Hair Color by Blunting Methionine Sulfoxide Repair, The FASEB Journal, 23; 2065-2075 (July, 2009); published online before print (Feb. 23, 2009)). As a result of these events, melanogenesis is hindered, leading to gradual loss of hair color.

In order to defend themselves against such oxidative damages, cells naturally produce antioxidative agents such as superoxide dismutase and catalase. Superoxide dismutase is an enzyme that catalyzes the dismutation of harmful superoxide into oxygen and H₂O₂. Catalase then catalyzes the conversion of H₂O₂ to harmless constituents: water and oxygen. Thus, the collaborative actions of catalase and superoxide dismutase serve to prevent oxidative damages to cells. As the body produces less catalase with age, imbalance occurs in the relative concentrations of catalase and H₂O₂. This appears to trigger the inactivation of catalase, which contributes to the increase in the level of H₂O₂. In fact, the accumulation of H₂O₂ is observed on human gray/white hair shafts, along with reduced levels of catalase in the hair follicle. (id. Par. 7). This finding suggests that the accumulation of H₂O₂ may be triggered by body's insufficient production of catalase.

In light of the foregoing, the focus is now turned to ways in which graying of hair can be prevented or reversed by undoing the accumulation of H₂O₂. One such solution is to increase the level of catalase by either supplying or inducing the production of catalase. This way, the amount of H₂O₂ can be reduced to a normal level. Continuous supply of catalase to the affected location would help maintain the catalase level at a sufficient concentration and prevents re-accumulation of H₂O₂. Intravenous injection or topical application of catalase is undesirable, as these methods could be inconvenient, ineffective, and/or unsafe.

Orally administered catalase is currently available on the market, including “Progena Hepato Plus 60” available from Meditrend, Inc. (Albuquerque, N. Mex.) and “BioGuard plus” available from Imagenetix Corporation (San Diego, Calif.). However, the effect of orally administered catalase without proper enhancing agents is inadequate to prevent graying of hair. Thus, there is a clear need for providing a dietary composition that overcomes these and other drawbacks of previous solutions, and which provides a synergistic composition that is safe and effective to use.

In search for a more effective form of catalase, it has been unexpectedly discovered that the effect of orally administered catalase is synergistically enhanced when catalase is provided in combination with copper, vitamin B6, and biotin. Moreover, it has also been discovered that when additional agents including para-aminobenzoic acid, folic acid, zinc, and pantothenic acid and certain classes of ingredients such as 5-α-reductase inhibitors and antioxidants are orally administered together with the aforementioned composition, the beneficial effects on hair are considerably amplified.

### SUMMARY OF INVENTION

It is therefore an object of the invention to provide a dietary composition comprising catalase and a synergistic core component comprising copper, vitamin B6, and biotin.

It is yet an object of the invention to provide a dietary composition comprising catalase and a synergistic core component comprising copper, vitamin B6, biotin, para-aminobenzoic acid, folic acid, zinc, and pantothenic acid.

It is another object of the invention to provide a dietary composition that promotes healthy hair in humans comprising catalase and a synergistic core component comprising copper, vitamin B6, biotin, para-aminobenzoic acid, folic acid, zinc, and pantothenic acid.

It is another object of the invention to provide a dietary composition that is useful in retarding, preventing, and/or reversing the graying of hair comprising catalase and a synergistic core component comprising copper, vitamin B6, biotin, para-aminobenzoic acid, folic acid, zinc, and pantothenic acid.

It is yet another object of the present invention to enhance the anti-graying effect of catalase on hair by orally providing catalase with copper, vitamin B6, biotin, para-aminobenzoic acid, folic acid, zinc, and pantothenic acid.

It is another object of the invention to provide a dietary composition comprising 5α-reductase inhibitors, catalase, and a synergistic core component comprising copper, vitamin B6, biotin, para-aminobenzoic acid, folic acid, zinc, and pantothenic acid.

It is another object of the invention to provide a dietary composition that promotes healthy hair comprising 5α-reductase inhibitors, catalase, and a synergistic core component comprising copper, vitamin B6, biotin, para-aminobenzoic acid, folic acid, zinc, and pantothenic acid.

It is yet another object of the invention to provide a dietary composition is useful in retarding, preventing, and/or reversing the graying of hair comprising 5α-reductase inhibitors, catalase, and a synergistic core component comprising copper, vitamin B6, biotin, para-aminobenzoic acid, folic acid, zinc, and pantothenic acid.

It is another object of the invention to provide a dietary composition comprising an antioxidant component, catalase, and a synergistic core component comprising copper, vitamin B6, biotin, para-aminobenzoic acid, folic acid, zinc, and pantothenic acid.

It is another object of the invention to provide a dietary composition that promotes healthy hair comprising an antioxidant component, catalase, and a synergistic core component comprising copper, vitamin B6, biotin, para aminobenzoic acid, folic acid, zinc, and pantothenic acid.

It is yet another object of the invention to provide a dietary composition that is useful in retarding, preventing, and/or reversing the graying of hair comprising an antioxidant component, catalase, and a synergistic core component comprising copper, vitamin B6, biotin, para-aminobenzoic acid, folic acid, zinc, and pantothenic acid.

It is yet another object of the invention to provide a dietary composition that is useful for retarding, preventing, and/or reversing the graying of hair comprising L-tyrosine, 5-α-reductase inhibitors, catalase, and a synergistic core component comprising copper, vitamin B6, biotin, para-aminobenzoic acid, folic acid, zinc, and pantothenic acid.

It is yet another object of the invention to provide a dietary composition that is useful in retarding, preventing, and/or reversing the graying of hair comprising. L-tyrosine, an antioxidant component, catalase, and a synergistic core component comprising copper, vitamin B6, biotin, para-aminobenzoic acid, folic acid, zinc, and pantothenic acid.

It is yet another object of the invention to provide a dietary composition that is useful in retarding, preventing, and/or reversing the graying of hair comprising L-tyrosine, an antioxidant component, 5α-reductase inhibitors, catalase, and a synergistic core component comprising copper, vitamin B6, biotin, para-aminobenzoic acid, folic acid, zinc, and pantothenic acid.

It is yet another object of the invention to provide a method of retarding, preventing, and/or reversing the graying of hair by orally administering a dietary composition comprising L-tyrosine, an antioxidant component, 5α-reductase inhibitors, catalase, and a synergistic core component comprising copper, vitamin B6, and biotin.

It is yet another object of the invention to provide a method of retarding, preventing, and/or reversing the graying of hair by orally administering a dietary composition comprising L-tyrosine, an antioxidant component, 5α-reductase inhibitors, catalase, and a synergistic core component comprising copper, vitamin B6, biotin, para-aminobenzoic acid, folic acid, zinc, and pantothenic acid.

The present invention relates to a dietary composition comprising approximately 2,500 IU to approximately 10,000 IU per day of catalase, a synergistic core component, 5α-reductase inhibitors, an antioxidant component, and approximately 100 mg to approximately 300 mg per day of L-tyrosine. Preferably, the synergistic core component comprises sufficient copper oxide to supply approximately 1 mg to approximately 3 mg per day of copper, approximately 5 mg to approximately 20 mg per day of vitamin B6, approximately 200 mcg to approximately 600 mcg per day of biotin, approximately 100 mg to approximately 400 mg per day of para-aminobenzoic acid, approximately 200 mcg to approximately 600 mcg per day of folic acid, sufficient zinc oxide to supply approximately 5 mg to approximately 20 mg per day of zinc, approximately 50 mg to approximately 400 mg per day of pantothenic acid. Further, 5α-reductase inhibitors preferably comprise approximately 100 mg to approximately 600 mg per day of Saw palmetto, approximately 50 mg to approximately 300 mg per day of phytosterols (45% beta-sitosterol), and approximately 50 mg to approximately 250 mg per day of nettle root extract. The antioxidant component preferably comprises approximately 50 mg to approximately 300 mg per day of horsetail, approximately 5 mg to approximately 20 mg per day of Fo-Ti, approximately 5 mg to approximately 20 mg per day of chlorophyll, and approximately 5 mg to approximately 30 mg per day of barley grass.

### DETAILED DESCRIPTION OF THE INVENTION

A detailed illustrative embodiment of the present invention is disclosed herein. However, the present invention may be embodied in a wide variety of forms, some of which may be quite different from those in the disclosed embodiment. Consequently, the specific ingredients and functional details disclosed herein are merely representative, yet in that regard, they are deemed to afford the best embodiment for purposes of disclosure and to provide a basis for the claims herein which define the scope of the present invention.

Moreover, well known methods and procedures for both carrying out the objectives of the present invention and illustrating the preferred embodiment are incorporated herein by reference but have not been described in detail as not to unnecessarily obscure aspects of the present invention.

The dietary composition according to the present invention is useful for retarding, preventing, and/or reversing the graying of hair of a user. A potent combination of ingredients in the present invention is designed to reduce the buildup of H₂O₂ and promote melanogenesis in the hair follicles, thereby restoring pigments in newly grown hair. Such anti-graying effects of the dietary composition are more effective and amplified than those of individual ingredients taken alone. Furthermore, the dietary composition of the present invention may promote healthy hair growth with more volume, luster, and strength. Individuals whose hair has already turned gray may take the dietary composition daily to improve the quality of hair as well as retard, prevent, and/or reverse the graying of hair without the use of hair coloring/bleaching. Those whose hair has not turned gray may take the dietary composition to improve the quality of hair as well as retard and/or prevent the onset of gray hair.

The dietary composition of the present invention is an orally administered composition. It provides a safer and more convenient alternative to hair coloring/bleaching, as it requires no messy preparation, clean up, touch up, or harsh chemicals. Unlike the time-consuming process of coloring/bleaching, the oral administration of the dietary composition can be performed within seconds. The dietary composition is safe enough to be taken daily or only at times when a user wishes to do so. However, it is preferred that the user consumes the predetermined dosage of the dietary composition everyday for maximum results. Unless specified otherwise, the amount or range of each ingredient in the dietary composition given throughout this disclosure is a recommended amount per day. Naturally, dose levels can be adjusted to provide the recommended amount per day in a reasonable number of units (e.g., two-three capsules per day).

The dietary composition of the present invention can be prepared into various forms including, but not limited to liquid, gel, gelatin capsule, powder, bar, capsule, tablet, or the like. Preferably, the dietary composition of the present invention is provided in the form of a capsule. Preferably, the user will take the capsule in the conventional manner with a glass of liquid (e.g., water, juice, or the like) with meals.

The ingredients used in the dietary composition according to the present invention are considered generally safe and natural. The dietary composition strategically combines catalase with other ingredients so as to synergistically enhance the anti-graying effects on hair. Preferably, catalase is present in the dietary composition in the amount of between approximately 2,500 International Units (IU) and approximately 10,000 IU, more preferably approximately 5,000 IU. One IU of catalase converts 1 μM (micromole) of H₂O₂ to water and oxygen per minute at pH 7.0 and 25° C.

Catalase acts to reduce the buildup of H₂O₂ and allows for melanocytes to resume melanogenesis. Catalase also complements the actions of superoxide dismutase, and thus, promotes a more efficient process of antioxidative activities. Catalase has the highest substrate turnover rate of all enzymes; one molecule of catalase can decompose millions of molecules of H₂O₂ to water and oxygen per second. Preferable sources of catalase include catalase purified from bovine liver, fractionated liver, bacteria, Aspergillus niger, and plants.

The dietary composition of the present invention further comprises a synergistic core component. The synergistic core component directly or indirectly interacts with catalase to synergistically enhance its effects. The ingredients in the core component were meticulously selected and tested for their ability to enhance the effects of catalase and complement the potency of one another.

The synergistic core component of the dietary composition comprises approximately 1 mg to approximately 3 mg of copper. Most preferably, the dietary composition comprises approximately 1 mg of copper. Copper as used in the present invention can be provided in any pharmaceutically acceptable forms including copper oxides, salts, and/or complexes thereof. Most preferably, copper is provided in the form of copper oxide, such as cuprous oxide or cupric oxide.

Copper interacts synergistically with catalase to retard, prevent, and/or reverse the graying of hair in a user. Specifically, approximately 2,500 IU to approximately 10,000 IU of catalase combined with copper in the dietary composition is crucial for the successful outcome of the present invention.

It is known that trace elements including copper are lower in gray hair than in black hair. (Tsai Yung-Yueh, et al., Concentrations of Potassium, Sodium, Magnesium, Calcium, Copper, Zinc, Manganese and Iron in Black and Gray Hairs, Journal of Health Science, 46(1); 46-48 (2000)). The observation suggests that reduction of copper in hair is one of the factors associated with hair graying. Moreover, copper is a known cofactor for superoxide dismutase. Thus, copper doubly acts against oxidative damages of cells and biomolecules by combating H₂O₂ before and after its formation.

Preferably, the synergistic core component of the dietary composition further comprises approximately 5 mg to approximately 20 mg of vitamin B6, more preferably approximately 10 mg of vitamin B6. Vitamin B6 interacts synergistically with catalase to retard, prevent, and/or reverse the graying of hair in a user. Vitamin B6 is a water-soluble vitamin which can exist in various forms, seven of which are known to date: pyridoxine, pyridoxine 5′-phosphate; pyridoxal; pyridoxal 5-phosphate; pyridoxamine; pyridoxamine 5′-phosphate; and 4-pyridoxic acid. pyridoxal 5-phosphate, the metabolically active form of vitamin B6, is involved in many aspects of macronutrient metabolism, neurotransmitter synthesis, histamine synthesis, hemoglobin synthesis and function and gene expression. Preferred forms of vitamin B6 in the present invention include, but are not limited to pyridoxine hydrochloride, pyridoxal 5-phosphate, calcium pyridoxine 5-phosphate, and pyridoxal.

Preferably, the synergistic core component of the dietary composition further comprises approximately 200 mcg to approximately 600 mcg of biotin, more preferably approximately 300 mcg of biotin. Biotin interacts synergistically with catalase to retard, prevent, and/or reverse the graying of hair in a user. Biotin, also known as vitamin H, is a water-soluble vitamin and an essential coenzyme in the metabolism of fatty acids and amino acids. Biotin also plays a role in the citric acid cycle, a crucial process in the generation of biochemical energy in aerobic respiration. It is known that biotin deficiency causes hair loss and/or discoloration of hair. This is often reversible with biotin supplementation as demonstrated by Innis and Allardyce (Sheila M. Innis, et al., Possible Biotin Deficiency in Adults Receiving Long-Term Total Parenternal Nutrition, The American Journal of Clinical Nutrition, 37; 185-187 (February, 1983)).

Preferably, the synergistic core component of the dietary composition further comprises approximately 100 mg to approximately 400 mg of para-aminobenzoic acid (PABA), more preferably approximately 200 mg of PABA. PABA, also known as 4-aminobenzoic acid, interacts synergistically with catalase to retard, prevent, and/or reverse the graying of hair in a user. PABA is usually not considered essential for human survival, but plays a vital role in hair pigment restoration. For decades, many studies have been reporting that PABA helps to restore hair to its natural color. For example, Sieve reported in 1941 that daily administration of PABA for two months resulted in marked darkening of the hair in 50 patients with gray hair. (B. F. Sieve, Clinical Achromotrichia, Science, 94; (2437) 257-258 (Sep. 12, 1941)). In a separate study, Zarafonetis described 5 cases of dramatic hair color change and hair re-growth in 20 patients, who were taking varying amounts of PABA daily for other conditions. (Chris J. D. Zarafonetis, Darkening of Gray Hair During Para-Amino-Benzoic Acid Therapy, The Journal of Investigative Dermatology, 15; 399-401 (1950)). The effect of PABA on gray hair is believed to be effective when administered in large doses. As PABA is water soluble and can be excreted in the urine, high concentration of PABA is generally well-tolerated by a healthy individual. However, consumption of PABA without the supervision of a physician should be limited to less than 3 g per day.

Preferably, the synergistic core component of the dietary composition further comprises approximately 200 mcg to approximately 600 mcg of folic acid, more preferably approximately 400 mcg of folic acid. Folic acid interacts synergistically with catalase to retard, prevent, and/or reverse the graying of hair in a user. Folic acid is an essential water-soluble vitamin, as it is necessary for the synthesis of RNA and DNA, cell growth and division. As the human body can make PABA from folic acid, consumption of folic acid may provide a long-term supply of PABA to the user's body.

Preferably, the synergistic core component of the dietary composition further comprises approximately 5 mg to approximately 20 mg of zinc, more preferably approximately 10 mg of zinc. Zinc as used in the present invention can be provided in any acceptable forms including zinc oxides, salts, or complexes thereof. Preferably, zinc is provided in the form of zinc oxide. Zinc interacts synergistically with catalase to retard, prevent, and/or reverse the graying of hair of a user. Zinc is an essential trace element for many living organisms including human. In humans, zinc interacts with a wide range of biomolecules to regulate biological processes including the metabolism of RNA and DNA, signal transduction, and gene expression. It is estimated that about 10% human proteins require direct interaction with zinc, in addition to those that transport zinc. Similar to copper, zinc is a known cofactor for superoxide dismutase. Zinc also plays an essential role in melanogenesis in hair. This is evidenced by the presence of a zinc reservoir in melanocytes, cells that perform melanogenesis. Zinc is believed to facilitate melanogenesis by binding to various biomolecules involved in melanogenesis including melanin, metallothionein, and melanotransferrin. (J. Borovanský, Zinc in Pigmented Cells and Structures, Interactions and Possible Roles, Sborník léka\check{r}ský, 95(4); 309-320 (1994)). Furthermore, zinc is believed to promote healthy hair and hair growth. Zinc deficiency in humans has been shown to produce hair thinning, baldness, and irritated scalp. These symptoms are reversible with proper oral supplementation of zinc.

Preferably, the synergistic core component of the dietary composition further comprises approximately 50 mg to approximately 400 mg of pantothenic acid, more preferably approximately 300 mg of pantothenic acid. Pantothenic acid, also called vitamin B5, interacts synergistically with catalase to retard, prevent, and/or reverse the graying of hair in a user. Pantothenic acid is a water-soluble vitamin. It is essential to sustain life, as it is indispensible to the metabolism and synthesis of carbohydrates, fats, and proteins. Doses of 2 g/day of pantothenic acid in the form of calcium pantothenate may reduce the duration of morning stiffness, degree of disability, and pain severity in rheumatoid arthritis patients. Supplementation of pantothenic acid may improve oxygen utilization efficiency and reduce lactic acid accumulation in athletes, resulting in increased energy. Furthermore, it is believed that pantothenic acid is an “anti-gray hair factor” and its incorporation in human diet inhibits premature graying of hair. (U.S. Pat. No. 2,406,741)

The derivative of pantothenic acid, pantothenol, is a more stable form of the vitamin and is often used as a source of the vitamin in multivitamin supplements. Another common supplemental form of the vitamin is calcium pantothenate. Calcium pantothenate is often used in dietary supplements because as a salt, it is more stable than pantothenic acid in the digestive tract allowing for better absorption.

In one embodiment, the synergistic core component comprises sufficient copper oxide to supply approximately 1 mg to approximately 3 mg of copper, approximately 5 mg to approximately 20 mg of vitamin B6, and approximately 200 mcg to approximately 600 mcg of biotin. More preferably, the synergistic core component comprises sufficient copper oxide to supply approximately 1 mg to approximately 3 mg of copper, approximately 5 mg to approximately 20 mg of vitamin B6, approximately 200 mcg to approximately 600 mcg of biotin, approximately 100 mg to approximately 400 mg of PABA, approximately 200 mcg to approximately 600 mcg of folic acid, sufficient zinc oxide to supply approximately 5 mg to approximately 20 mg of zinc, and approximately 50 mg to approximately 400 mg of pantothenic acid. Most preferably, the synergistic core component comprises sufficient copper oxide to supply approximately 1 mg of copper, approximately 10 mg of vitamin B6, approximately 300 mcg of biotin, approximately 200 mg of PABA, approximately 400 mcg of folic acid, sufficient zinc oxide to supply approximately 10 mg of zinc, and approximately 300 mg of pantothenic acid.

The dietary composition of the present invention further comprises 5α-reductase inhibitors. 5α-reductase inhibitors block the action of 5α-reductase, an enzyme that catalyzes the conversion of testosterone into dihydrotestosterone, a more potent form of testosterone. Excess dihydrotestosterone resulting from overactive 5α-reductase is believed to cause androgenetic alopecia, a common form of hair loss in both male and female humans. The 5α-reductase inhibitors work synergistically with catalase and the core component to promote thick and healthy hair. Furthermore, 5-α-reductase inhibitors enhance the pigment restoration effect of catalase and the core component.

Preferably, the 5α-reductase inhibitors of the dietary composition comprises approximately 100 mg to approximately 600 mg of Saw palmetto, more preferably approximately 300 mg of Saw palmetto. Most preferably, the 5-α-reductase inhibitors comprise approximately 300 mg of Saw palmetto, of which at least approximately 40% w/w to approximately 45% w/w is an extract of Saw palmetto. Saw palmetto interacts synergistically with catalase and the core component to retard, prevent, and/or reverse the graying of hair of a user. Saw palmetto has various health benefits and therapeutic properties including expectorant and antiseptic effects. Saw Palmetto extract refers to an extract of the fruit of Serenoa repens. It is highly enriched with fatty acids and phytosterols such as beta-sitosterol. Some experts believe that a preparation called lipidosterolic extract of Serenoa repens may cause fewer side effects. Saw palmetto extract is one of the popular alternative medicines taken for benign prostatic hyperplasia and androgenetic alopecia due to its 5α-reductase inhibitory activity. (Nelson Prager, et al., A Randomized, Double-Blind, Placebo-Controlled Trial to Determine the Effectiveness of Botanically Derived Inhibitors of 5AR in the Treatment of Androgenetic Alopecia, The Journal of Alternative and Complementary Medicine, 8(2); 143-152 (April, 2002)). Saw palmetto and an extract thereof as used in the present invention are prepared in a conventional manner known to those skilled in the art.

Preferably, the 5α-reductase inhibitors of the dietary composition further comprises approximately 50 mg to approximately 300 mg of phytosterols, more preferably approximately 100 mg of phytosterols. Phytosterols, also known as plant sterols, interact synergistically with catalase and the core component to retard, prevent, and/or reverse the graying of hair of a user. Phytosterols are a group of steroid alcohols that are naturally occurring in plants. Phytosterols function as essential structural components in the cell membrane. Phytosterols are found in small quantities in vegetable oils, especially sea buckthorn oil, corn oil, and soybean oil. Consumption of phytosterols is a common and highly recommended practice to promote overall health by medical and nutritional experts. According to the Food and Drug Administration (FDA), consuming at least 0.8 gram daily of phytosterols from a food source as part of a diet low in saturated fat and cholesterol may reduce the risk of heart disease. In fact, studies show that sterols can reduce cholesterol in human subjects by 10 to 15%. (Marie-Pierre St-Onge, et al., Consumption of a Functional Oil Rich in Phytosterols and Medium-Chain Triglyceride Oil Improves Plasma Lipid Profiles in Men, The Journal of Nutrition, 133; 1815-1820 (June, 2003)).

In addition to such health benefits, certain types of phytosterols have been shown to synergistically enhance the effects of Saw palmetto. Specifically, when beta-sitosterol, a type of phytosterol, is administered together with Saw Palmetto, healthy hair growth in individuals with mild to moderate hair loss was observed. (Nelson Prager, et al., A Randomized, Double-Blind, Placebo-Controlled Trial to Determine the Effectiveness of Botanically Derived Inhibitors of 5AR in the Treatment of Androgenetic Alopecia, The Journal of Alternative and Complementary Medicine, 8(2); 143-152 (April, 2002)).

Preferred types of phytosterols that are used in the present invention include, but are not limited to beta-sitosterol, campesterol, stigmasterol, brassicasterol, and ergosterol. Most preferably, the phytosterols used in the present invention comprise at least approximately 45% beta-sitosterol. Phytosterols as used in the present invention are prepared in a conventional manner known to those skilled in the art.

Preferably, the 5α-reductase inhibitors of the dietary composition further comprises approximately 50 mg to approximately 250 mg of nettle root extract, more preferably approximately 100 mg of nettle root extract. Nettle root extract interacts synergistically with catalase and the core component to retard, prevent, and/or reverse the graying of hair of a user. “Nettle” is a common name for Urtica dioica, an herbaceous perennial flowering plant also known as the Stinging Nettle. Nettle root extract is known to inhibit the activities of 5α-reductase. (James Occhiogrosso, Your Prostate, Your Libido, Your Life, Glenbridge Publishing Ltd., 19923 E. Long Ave. Centennial, Colo. 80016, pg. 104 (2007)). An extract from nettle root as used in the present invention is obtained in a conventional manner known to those skilled in the art.

Preferably, the 5α-reductase inhibitors are derived from natural sources, such as plants or fruits. In addition to aforementioned examples, such natural 5α-reductase inhibitors include, but are not limited to green tea extract, L-lysine, Linolenic acid, Pygeum africanum extract, and pumpkin seed oil. Most preferably, the 5α-reductase inhibitors of the present invention comprise approximately 100 mg to approximately 600 mg of Saw palmetto, approximately 50 mg to approximately 300 mg of phytosterols, and approximately 50 mg to approximately 250 mg of nettle root extract. More preferably, the 5α-reductase inhibitors of the present invention comprise approximately 300 mg of Saw palmetto, approximately of 100 mg of phytosterols, and approximately 100 mg of nettle root extract.

The dietary composition of the present invention further comprises an antioxidant component. The antioxidant component works synergistically with catalase and the core component to promote thick and healthy hair. Furthermore, the antioxidant component enhances the pigment restoration effect of catalase and the core component. The antioxidant component preferably comprises ingredients that are known to provide benefits specifically to hair.

Preferably, the antioxidant component comprises approximately 50 mg to approximately 300 mg of horsetail, more preferably approximately 100 mg of horsetail. Most preferably, the antioxidant component comprises approximately 100 mg of horsetail, of which at least approximately 7% w/w is an extract of horsetail. Horsetail interacts synergistically with catalase and the core component to retard, prevent, and/or reverse the graying of hair of a user. Horsetail refers to plants of the genus Equisetum. Equisetum is often called a “living fossil,” as it is a single surviving genus in the entire class of Equisetopsida. Due to its health benefits, the consumption of horsetail has been a common practice around the world. The young horsetail stems bearing strobili of some species are cooked and eaten by humans in Japan. The people of ancient Rome would also eat meadow horsetail in this manner, but they also used horsetail to make tea and thickening powder. Due to the presence of relatively large amounts of silica in the form of silicic acid and silicates in horsetail, its extract is often used to provide silica for supplementation. Water and ethanol extracts of field horsetail have been shown to have remarkable antioxidative activities. (Takeshi Nagai, et al., Antioxidative Activities of Water Extract and Ethanol Extract from Field Horsetail (Tsukushi) Equisetum Arvense L., Food-Chemistry, 91(3); 389-394 (July, 2005)). Horsetail is rich in essential nutrients including vitamins C and E, copper, and zinc, all of which are known to have or aid in antioxidative activities. Horsetail has been used for centuries as a remedy to treat many ailments including hair loss. Horsetail and an extract thereof as used in the present invention are prepared in a conventional manner known to those skilled in the art.

Preferably, the antioxidant component comprises approximately 5 mg to approximately 20 mg of Fo-Ti, more preferably approximately 10 mg of Fo-Ti. Fo-Ti interacts synergistically with catalase and the core component to retard, prevent, and/or reverse the graying of hair of a user. Fo-Ti, also known as Fo-ti-teng, Chinese knotweed, or Flowery knotweed, is a plant indigenous to China. Fo-Ti refers to a plant of the species Polygonum Multiflorum. Fo-Ti has been used in Chinese medicine for hundreds of years due to its “anti-aging” effects. The Chinese refer to Fo-Ti as “Heshouwu,” which translates to “black-haired Mr. He.” The name comes from a Chinese legend about an old villager, Mr. He. As a result of regular Fo-Ti consumption, Mr. He restored his sexual vitality and youthful appearance, including his hair color.

Dietary Fo-Ti can supply abundance of phospholipids such as phosphatidylcholine, phophatidylinositol, and phosphatidylethanolamine (cephalin). Phosphatidylcholine in particular, tends to decrease in number with age, and many experts recommend daily supplementation of phosphatidylcholine as an anti-aging measure. Furthermore, the root of Fo-Ti has chemical compounds similar in nature to resveratrol, a substance that is currently a focus of intense research for its antioxidative and anti-aging effects. Fo-Ti is believed to have restorative effects on graying hair, hair loss, and premature aging. Fo-Ti may be replaced with or supplemented by Japanese knotweed (Polygonum cuspidatum), which is also a source of resveratrol. Fo-Ti may be prepared as powder, tincture, or the like. Preferably, Fo-Ti is prepared into a powder form in a conventional manner known to those skilled in the art.

Preferably, the antioxidant component comprises approximately 5 mg to approximately 30 mg of barley grass, more preferably approximately 10 mg of barley grass. Barley grass interacts synergistically with catalase and the core component to retard, prevent, and/or reverse the graying of hair of a user. Barley is a grain plant that is widely used in the preparation of food for both humans and animals. Barley grass is the leaf portion of barley that remains after the seeds have been removed. Barley generally refers to plants of the genus Hordeum, but Hordeum vulgare and Hordeum distichon are perhaps the most widely used barley. Other Hordeum species may also be useful for the dietary supplement of the present invention.

Barley is a potent antioxidant. Many studies report significant antioxidant activities of barley, especially extracts of Hordeum vulgare L. (Zhao H., et al., Effects of Extraction Solvent Mixtures on Antioxidant Activity Evaluation and Their Extraction Capacity and Selectivity for Free Phenolic Compounds in Barley (Hordeum vulgare L.), Journal of Agricultural and Food Chemistry, 54(19); 7277-7286 (Sep. 20, 2006)). (Pin-Der Duh, et al., Antioxidant Effects of Water Extracts from Barley (Hordeum vulgare L.) Prepared under Different Roasting Temperatures, Journal of Agricultural and Food Chemistry, 49(3); 1455-1463 (Feb. 10, 2001)). Furthermore, a clinical study reported by Yu et al. suggests that oral supplementation of barley grass extract may help to scavenge free radicals. (Yu Y. M., et al., Effects of Young Barley Leaf Extract and Antioxidative Vitamins on LDL oxidation and Free Radical Scavenging Activities in Type 2 Diabetes, Diabetes and Metabolism, 28(2); 107-114 (April, 2002)). Barley grass as used in the present invention is obtained in a conventional manner known to those skilled in the art.

Preferably, the antioxidant component comprises approximately 5 mg to approximately 20 mg of chlorophyll, more preferably approximately 10 mg of chlorophyll. Chlorophyll interacts synergistically with catalase and the core component to retard, prevent, and/or reverse the graying of hair of a user. Dietary chlorophyll derivatives prevalent in both fresh and processed foods as well as dietary supplements have antioxidant and antimutagenic activities. (M. G. Ferruzzi, et al., Antioxidant and Antimutagenic Activity of Dietary Chlorophyll Derivatives Determined by Radical Scavenging and Bacterial Reverse Mutagenesis Assays, Journal of Food and Science, 67(7); 2589-2595 (Jul. 20, 2006)). Dietary chlorophyll is predominantly composed of lipophilic derivatives including chlorophyll a and b (fresh fruits and vegetables), metal-free pheophytins and pyropheophytins (thermally processed fruits and vegetables), as well as Zn-pheophytins and Zn-pyropheophytins (thermally processed green vegetables). Water-soluble derivatives including chlorophyllides, pheophorbides, as well as a commercial-grade derivative known as sodium copper chlorophyllin also contribute to the diversity of dietary chlorophyll derivatives. Chlorophyll as used in the present invention is obtained in a conventional manner known to those skilled in the art.

Other sources of antioxidants include, but are not limited to Camellia sinensis (green tea) extract, Pyrus malus (apple) fruit extract, Quercus rubra (red oak) bark extract, cinnamon bark extract, and Rheum palmatum (Chinese rhubarb) extract, ginkgo biloba, lemon grass extract, basil extract, grape seeds extract, rosemary extract, sage extract, thyme extract, chamomile extract, lavender extract, horse chestnut extract, and lemon extract. These plant/fruit extracts aid in the synergic anti-graying effects of the composition.

In one embodiment of the invention, the antioxidant component comprises approximately 50 mg to approximately 300 mg of horsetail, approximately 5 mg to approximately 20 mg of Fo-Ti, approximately 5 mg to approximately 20 mg of chlorophyll or its derivatives, and approximately 5 mg to approximately 30 mg of barley grass. More preferably, the antioxidant component comprises approximately of 100 mg of horsetail, approximately 10 mg of Fo-Ti, approximately 10 mg of chlorophyll or its derivatives, and approximately 10 mg of barley grass.

Preferably, the dietary composition of the present invention further comprises approximately 100 mg to approximately 300 mg of an amino acid, L-tyrosine (abbreviated as Tyr or Y), more preferably approximately 200 mg of L-tyrosine. Since L-amino acids represent the vast majority of amino acids found in proteins, L-tyrosine is preferred in the present invention and will be synonymous with “tyrosine” throughout this disclosure.

Tyrosine, also known as 4-hydroxyphenylalanine, works synergistically with catalase and the core component to enhance the pigment restoration effect of catalase and the core component. Tyrosine is a precursor to melanin, pigments that impart color to hair and skin. Specifically, eumelanin and pheomelanin require tyrosine for their biosynthesis. In fact, it has been shown that the amount of Tyrosine in a medium containing melanocyte culture strongly influences production of both eumelanin and pheomelanin. (Nico P. M. Smit, et al., Melanogenesis in Cultured Melanocytes can be Substantially Influenced by L-Tyrosine and L-Cysteine, The Journal of Investigative Dermatology, 109(6); 796-800 (December, 1997)). According to the study, a high concentration of tyrosine was always connected with increased pigmentation. This suggests that it is vital for hair melanocytes to have an ample supply of tyrosine for melanin production. A daily oral dosage generally recommended by World Health Organization is about 25 mg/kg body weight for an adult. It is not recommended to exceed 12 g per day.

The dietary composition of the present invention may also contain other ingredients or excipients that are commonly used in oral supplements known in the art. Such ingredients include, but are not limited to colorants, flavoring agents, binders/fillers, buffers, and preservatives.

The dietary composition of the present invention may further comprise one or more colorants. Non-limiting examples of colorants include colorants approved for use in food, drugs and cosmetics (FD&C) such as FD&C Blue No. 1, 2; FD&C Green No. 1, 3; FD&C Red No. 3, 40; FD&C Yellow No. 5, 6; paprika; beet red powder, and the like.

The dietary composition of the present invention may further comprise one or more flavoring agents. Non-limiting examples of flavoring agents include Apple watermelon PFC 9887, Apricot 23067 and 24829, Cherry 57.679/A, 213, 349, 107026, DP300684, WL-4658; Vanilla 28156; Strawberry 133.5655, 17C56217, and the like.

The dietary composition of the present invention may further comprise one or more binders/fillers. Non-limiting examples of binders/fillers include maltodextrin, lactose, dibasic calcium phosphate, sucrose, corn starch, magnesium stearate, microcrystalline cellulose, hydroxypropyl methylcellulose, and the like.

The dietary composition of the present invention may further comprise one or more buffers. Non-limiting examples of buffers include potassium metaphosphate, potassium phosphate, monobasic sodium acetate, sodium citrate and the like.

The dietary composition of the present invention may further comprise one or more preservatives. Non-limiting examples of preservatives include disodium EDTA, benzalkonium chloride, benzyl alcohol, phenol, phenylethyl alcohol, cetylpyridinium chloride, chlorobutanol, and the like.

The following examples further describe and demonstrate embodiments within the scope of the invention. The examples are given solely for the purpose of illustration and are not to be construed as limitations of the present invention, as many variations are possible without departing from the spirit and scope of the invention.

### Example 1

| Ingredients | Amount per day | |
|---|---|---|
| Catalase | 5,000 | IU |
| Copper (oxide) | 1 | mg |
| Vitamin B6 (pyridoxine hydrochloride) | 10 | mg |
| Biotin | 300 | mcg |
| PABA (para amino benzoic acid) | 200 | mg |
| Folic Acid | 400 | mcg |
| Zinc (oxide) | 10 | mg |
| Pantothenic Acid (calcium pantothenate) | 300 | mg |
| Horsetail (7% extract) | 100 | mg |
| Saw Palmetto (40-45% extract) | 300 | mg |
| Plant Sterols (45% beta-sitosterol) | 100 | mg |
| Fo Ti (powder) | 10 | mg |
| Nettle Root Extract | 100 | mg |
| Chlorophyll | 10 | mg |
| Barley Grass | 10 | mg |
| L-Tyrosine | 200 | mg |

### Example 2

| Ingredients | Amount Range per Day |
|---|---|
| Catalase | 2,500 IU - 10,000 IU |
| Copper (oxide) | 1 mg - 3 mg |
| Vitamin B6 (pyridoxine hydrochloride) | 5 mg - 20 mg |
| Biotin | 200 mcg - 600 mcg |
| PABA (para amino benzoic acid) | 100 mg - 400 mg |
| Folic Acid | 200 mcg - 600 mcg |
| Zinc (oxide) | 5 mg - 20 mg |
| Pantothenic Acid (calcium pantothenate) | 50 mg - 400 mg |
| Horsetail (7% extract) | 50 mg - 300 mg |
| Saw Palmetto (40-45% extract) | 100 mg - 600 mg |
| Plant Sterols (45% beta-sitosterol) | 50 mg - 300 mg |
| Fo Ti (powder) | 5 mg - 20 mg |
| Nettle Root Extract | 50 mg - 250 mg |
| Chlorophyll | 5 mg - 20 mg |
| Barley Grass | 5 mg - 30 mg |
| L-Tyrosine | 100 mg - 300 mg |

The dietary compositions as shown in Examples 1 and 2 can be prepared into various forms including, but not limited to liquid, bar, powder, gel, gelatin capsule, capsule, tablet, or the like. Preferably, the dietary composition as shown in Examples 1 or 2 is provided in the form of a capsule comprising gelatin, magnesium stearate, and silicon dioxide. Other acceptable carriers and/or excipients known in the art may also be used for the dietary composition.

It is contemplated that the user will take the dietary composition in the conventional manner with a glass of water. The dietary composition as shown in Examples 1 or 2 can be taken daily or only at times when the user wishes to do so, however, it is preferred for maximum benefit that the user consumes the amount as proposed in Examples 1 or 2 daily with meals. Furthermore, the dietary composition according to Examples 1 or 2 is most effective when its dosage is split into two portions such that a half of the daily amount is taken in the morning and the other half is taken in the evening.

It must also be noted that generally, the already exposed hair shaft cannot be colored by melanocytes, as melanogenesis occurs in hair follicles. Naturally, visible effects of the dietary supplement may not be observed until new growth of the hair occurs. Therefore, it typically takes about weeks to months for the effects of the dietary composition of the present invention to be noticed by the user, depending on the rate of hair growth.

### Example 3

### Demonstration of Pigment Restorative Effect of the Dietary Composition According to Example 1

A number of test subjects have taken the dietary composition as described in Example 1 for varying lengths of time. Summarized below are reported results based on the subjects' qualitative observation.

Subject No. 1—A 53 year old female took the dietary composition according to Example 1 in two portions daily for six weeks. At the beginning of the trial, the subject's hair was brown with enough gray hair to impart the “salt and pepper” effect. After three weeks, the subject noticed increase in her daily energy level as well as volume in her hair. By the sixth week, the subject stopped coloring her hair, as gray hair was no longer visible.

Subject No. 2—A 47 year old male took the dietary composition according to Example 1 in two portions daily for five weeks. Prior to the trial, the subject had patches of gray hair on both sides of his head. By the fifth week, the gray hair had virtually disappeared, replaced by new outgrowth of pigmented hair.

Subject No. 3—A 45 year old male took the dietary composition according to Example 1 in two portions for seven weeks daily. Prior to the trial, the subject's head and eyebrows were completely covered with gray hair. At the end of the seventh week, the subject's eyebrows and scalp hair were substantially darker.

While the present invention has been described with reference to the preferred embodiment and alternative embodiments, which have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

What is claimed is:

1. A dietary composition for promoting healthy hair growth in a user, said dietary composition comprising approximately 2,500 IU to approximately 10,000 IU per day of catalase and a synergistic core component; wherein said synergistic core component comprises copper, vitamin B6, and biotin.

2. A dietary composition according to claim 1, wherein the dietary composition promotes melanogenesis in hair of said user.

3. A dietary composition according to claim 1, wherein said synergistic core component further comprising one or more ingredients selected from the group consisting of para-aminobenzoic acid, folic acid, zinc, and pantothenic acid.

4. A dietary composition according to claim 1, further comprising at least one 5α-reductase inhibitor selected from the group consisting of Saw palmetto, phytosterols, nettle root extract, green tea extract, L-lysine, Linolenic acid, Pygeum africanum extract, and pumpkin seed oil.

5. A dietary composition according to claim 1, further comprising an antioxidant component selected from the group consisting of horsetail, Fo-Ti, chlorophyll, barley grass, green tea extract, apple fruit extract, Quercus rubra (red oak) bark extract, cinnamon bark extract, and Rheum palmatum (Chinese rhubarb) extract, ginkgo biloba, lemon grass extract, basil extract, grape seeds extract, rosemary extract, sage extract, thyme extract, chamomile extract, lavender extract, horse chestnut extract, and lemon extract, and mixtures thereof.

6. A dietary composition according to claim 1, further comprising an effective amount of L-tyrosine.

7. A dietary composition for promoting healthy hair growth in a user, said dietary composition comprising approximately 2,500 IU to approximately 10,000 IU per day of catalase, a synergistic core component, at least one 5α-reductase inhibitor, an antioxidant component, and an effective amount of L-tyrosine; wherein said synergistic core component comprises copper, vitamin B6, and biotin.

8. A dietary composition according to claim 7, wherein the dietary composition promotes melanogenesis in hair of said user.

9. A dietary composition according to claim 7, wherein said synergistic core component further comprising one or more ingredients selected from the group consisting of para-aminobenzoic acid, folic acid, zinc, and pantothenic acid.

10. A dietary composition according to claim 7, wherein at least one of said 5α-reductase inhibitor is selected from the group consisting of Saw palmetto, phytosterols, nettle root extract, green tea extract, L-lysine, Linolenic acid, Pygeum africanum extract, and pumpkin seed oil.

11. A dietary composition according to claim 7, wherein said antioxidant component is selected from the group consisting of horsetail, Fo-Ti, chlorophyll, barley grass, green tea extract, apple fruit extract, Quercus rubra (red oak) bark extract, cinnamon bark extract, and Rheum palmatum (Chinese rhubarb) extract, ginkgo biloba, lemon grass extract, basil extract, grape seeds extract, rosemary extract, sage extract, thyme extract, chamomile extract, lavender extract, horse chestnut extract, and lemon extract, and mixtures thereof.

12. A dietary composition for promoting healthy hair growth in a user comprising per day:
approximately 5,000 IU of catalase;
approximately 1 mg of copper;
approximately 10 mg of vitamin B6;
approximately 300 mcg of biotin;
approximately 200 mg of PABA;
approximately 400 mcg of folic acid;
approximately 10 mg of zinc;
approximately 300 mg of pantothenic acid;
approximately 100 mg of horsetail;
approximately 300 mg of Saw palmetto;
approximately 100 mg of Phytosterols;
approximately 10 mg of Fo-Ti powder;
approximately 100 mg of nettle root extract;
approximately 10 mg of chlorophyll;
approximately 10 mg of barley grass; and
approximately 200 mg of L-tyrosine.

13. A dietary composition according to claim 12, wherein the dietary composition promotes melanogenesis in hair of said user.

14. A dietary composition according to claim 12, wherein said dietary composition is divided into two portions; said two portions consist of a morning portion and an evening portion; said morning portion is ingested by said user in the morning of a day and said evening portion is ingested by said user in the evening of the same day.

15. A dietary composition for promoting healthy hair growth in a user comprising per day:
approximately 2,500 IU to approximately 10,000 IU of catalase;
approximately 1 mg to approximately 3 mg of copper;
approximately 5 mg to approximately 20 mg of vitamin B6;
approximately 200 mcg to approximately 600 mcg of biotin;
approximately 100 mg to approximately 400 mg of PABA;
approximately 200 mcg to approximately 600 mcg of folic acid;
approximately 5 mg to approximately 20 mg of zinc;
approximately 50 mg to approximately 400 mg of pantothenic acid;
approximately 50 mg to approximately 300 mg of horsetail;
approximately to 100 mg approximately 600 mg of Saw palmetto;
approximately 50 mg to approximately 300 mg of Phytosterols;
approximately 5 mg to approximately 20 mg of Fo-Ti powder;
approximately 50 mg to approximately 250 mg of nettle root extract;
approximately 5 mg to approximately 20 mg of chlorophyll;
approximately 5 mg to approximately 30 mg of barley grass; and
approximately 100 mg to approximately 300 mg of L-tyrosine.

16. A dietary composition according to claim 15, wherein the dietary composition promotes melanogenesis in hair of said user.

17. A method of promoting melanogenesis in hair of a user comprising:
a) providing a dietary composition comprising approximately 2,500 IU to approximately 10,000 IU per day of catalase and a synergistic core component, wherein said synergistic core component comprises copper, vitamin B6, biotin, para-aminobenzoic acid, folic acid, zinc, and pantothenic acid;
b) orally administering said dietary composition to said user daily;
c) releasing said catalase and said synergistic core component within said user's body, wherein said release of said catalase and said synergistic core component promotes melanogenesis in said hair of said user.

18. A method according to claim 17, wherein said dietary composition further comprises at least one 5α-reductase inhibitor.

19. A method according to claim 17, wherein said dietary composition further comprises an antioxidant component.

20. A method according to claim 17, wherein said dietary composition further comprises an effective amount of L-tyrosine.
